(19) United States
(12) Patent Application Publication
(10) Pub. No.: US 2022/0296398 A1
(43) Pub. Date: Sep. 22, 2022

(54) CONTROLLABLE PIERCING SIDE-BRANCH STENTS

(71) Applicant: BIFLOW Medical Ltd., Nazeret-Elit (IL)

(72) Inventor: Haim ACKERMAN, Caesarea (IL)

(73) Assignee: BIFLOW Medical Ltd., Nazeret-Elit (IL)

(21) Appl. No.: 17/620,743

(22) PCT Filed: Jun. 23, 2020
(86) PCT No.: PCT/IL2020/050695

§ 371 Date: Dec. 20, 2021

Related U.S. Application Data
(60) Provisional application No. 62/866,000, filed on Jun. 25, 2019.

(51) Int. Cl. A61F 2/915; A61F 2/07

(52) U.S. Cl. CPC A61F 2/915; A61F 2/07; A61F 2002/91558; A61F 2220/0033; A61F 2250/006; A61F 2002/061

(57) ABSTRACT

Side-branch, piercing stents and corresponding methods are provided. The stent body has apically controllably movable leaves that have a closed state and an open state, and at least one of the leaves has an apical piercing tip that is controllably moveable from a contracted state to an extended, piercing state, in which it pierces an apically adjacent element such as a main stent graft, an aneurism or a blood vessel, creates an opening therein, enables insertion of the apical tip of the side -branch stent and allows for spreading the leaves to anchor them within the element and ensure blood flow between the volume of the element and the side branches. Controlling element(s) control the movements of the tip(s) and the leaves to enable controlled and accurate positioning of the side-branch stent, its attachment to the main element and its anchoring therein.

### BACKGROUND OF THE INVENTION

### 1. Technical Field

The present invention relates to the field of side-branch stents, and more particularly, to controllable piercing side-branch stents.

### 2. Discussion of Related Art

U.S. Pat. No. 9,358,099, incorporated herein by reference in its entirety, teaches stent grafts provided for a fenestration to be created by a side branch fenestration creation device, and kits comprising a side branch fenestration creation device and a stent graft in which a fenestration may be created by the side branch vessel fenestration creation device, once deployed in an artery. The fenestration may be formed through a stent graft at a location where a main vessel (in which the stent graft is placed) branches into a side branch vessel. The side branch vessel stent may be placed in the side branch vessel so that at least a portion of the side branch vessel stent protrudes into the lumen of the main vessel; and the stent graft may be deployed in the main vessel, so that at least the portion of the side branch vessel stent punctures the stent graft at the location where the main vessel branches into the side branch vessel; or that the side branch vessel stent marks the location where the fenestration is to be formed, so that additional means may be inserted or may in advance have been inserted to aid in the creation of the fenestration.

### SUMMARY OF THE INVENTION

The following is a simplified summary providing an initial understanding of the invention. The summary does not necessarily identify key elements nor limit the scope of the invention, but merely serves as an introduction to the following description.

One embodiment of the present invention provides a side-branch, piercing stent comprising: a stent body having apically at least three movable leaves, the leaves configured to be controllably moveable in a medial direction between at least two leaf states comprising (i) a closed state in which an apical stent opening is smaller than a cross section of the stent, and (ii) an open state in which the apical stent opening is larger than the cross section of the stent, and at least one controlling member configured to control, mechanically and upon actuation, the movement of the leaves. At least one of the leaves is configured to have an apical piercing tip that is controllably moveable between at least two piercing states comprising (a) an extended state in which the apical piercing tip is positioned to pierce an element positioned adjacent to the apex of the stent, and (b) a contracted state in which the apical piercing tip is positioned not to pierce an element positioned adjacent to the apex of the stent, and the at least one controlling member is further configured to control, mechanically and upon actuation, the movement of the apical piercing tip of the at least one of the leaves.

One embodiment of the present invention provides a method of configuring a side-branch stent for attachment to an element positioned adjacent to the apex of the side-branch stent, the method comprising: configuring the side-branch stent body to have apically at least three movable leaves, the leaves configured to be controllably moveable in a medial direction between at least two leaf states comprising (i) a closed state in which an apical stent opening is smaller than a cross section of the stent, and (ii) an open state in which the apical stent opening is larger than the cross section of the stent, configuring at least one of the leaves to have an apical piercing tip that is controllably moveable between at least two piercing states comprising (a) an extended state in which the apical piercing tip is positioned to pierce the element positioned adjacent to the apex of the stent, and (b) a contracted state in which the apical piercing tip is positioned not to pierce the element positioned adjacent to the apex of the stent, and associating with the leaves at least one controlling member configured to control, mechanically and upon actuation, the movement of the leaves and the movement of the apical piercing tip of the at least one of the leaves.

One embodiment of the present invention provides a method of attaching a side-branch stent to an element positioned adjacent to the apex of the side-branch stent, the method comprising: moving, controllably, an apical piercing tip of at least one of the leaves into (a) an extended state in which the apical piercing tip is positioned to pierce the element positioned adjacent to the apex of the stent, and moving, controllably, at least three apical movable leaves in a medial direction from (i) a closed state in which an apical stent opening is smaller than a cross section of the stent into (ii) an open state in which the apical stent opening is larger than the cross section of the stent.

These, additional, and/or other embodiments and/or advantages of the present invention are set forth in the detailed description which follows; possibly inferable from the detailed description; and/or learnable by practice of the present invention.

### BRIEF DESCRIPTION OF THE DRAWINGS

For a better understanding of embodiments of the invention and to show how the same may be carried into effect, reference will now be made, purely by way of example, to the accompanying drawings in which like numerals designate corresponding elements or sections throughout.

In the accompanying drawings:

FIGS. 1A and 1B illustrate schematically the attachment of side-branch, piercing stents to elements positioned adjacent to the apexes of the stents, according to some embodiments of the invention.

FIGS. 2A and 2B and FIGS. 3A and 3B illustrate schematically side-branch, piercing stents, according to some embodiments of the invention.

FIG. 4 is a high-level flowchart illustrating methods, according to some embodiments of the invention.

### DETAILED DESCRIPTION OF THE INVENTION

In the following description, various embodiments of the present invention are described. For purposes of explanation, specific configurations and details are set forth in order to provide a thorough understanding of the present invention. However, it will also be apparent to one skilled in the art that the present invention may be practiced without the specific details presented herein. Furthermore, well known features may have been omitted or simplified in order not to obscure the present invention. With specific reference to the drawings, it is stressed that the particulars shown are by way of example and for purposes of illustrative discussion of the present invention only, and are presented in the cause of providing what is believed to be the most useful and readily understood description of the principles and conceptual embodiments of the invention. hi this regard, no attempt is made to show structural details of the invention in more detail than is necessary for a fundamental understanding of the invention, the description taken with the drawings making apparent to those skilled in the art how the several forms of the invention may be embodied in practice.

Before at least one embodiment of the invention is explained in detail, it is to be understood that the invention is not limited in its application to the details of construction and the arrangement of the components set forth in the following description or illustrated in the drawings. The invention is applicable to other embodiments that may be practiced or carried out in various ways as well as to combinations of the disclosed embodiments. Also, it is to be understood that the phraseology and terminology employed herein are for the purpose of description and should not be regarded as limiting.

Embodiments of the present invention provide efficient and economical methods and mechanisms for attaching side-branch stents to main stent grafts, aneurism and/or blood vessels, and thereby provide improvements to the technological field of aneurism and blood vessel treatment. Side-branch, piercing stents are provided, as well as methods of configuring and using the stents. The stent body has apically controllably movable leaves (or fingers) that have a closed state and an open state, and at least one of the leaves has an apical piercing tip that is controllably moveable from a contracted state to an extended, piercing state, in which it pierces an apically adjacent element such as a main stent graft, an aneurism or a blood vessel, creates an opening therein, enables insertion of the apical tip of the side-branch stent and allows for spreading the leaves to anchor them within the element and ensure blood flow between the volume of the element and the side branches. Controlling element(s) control the movements of the tip(s) and the leaves to enable controlled and accurate positioning of the side-branch stent, its attachment to the main element and its anchoring therein.

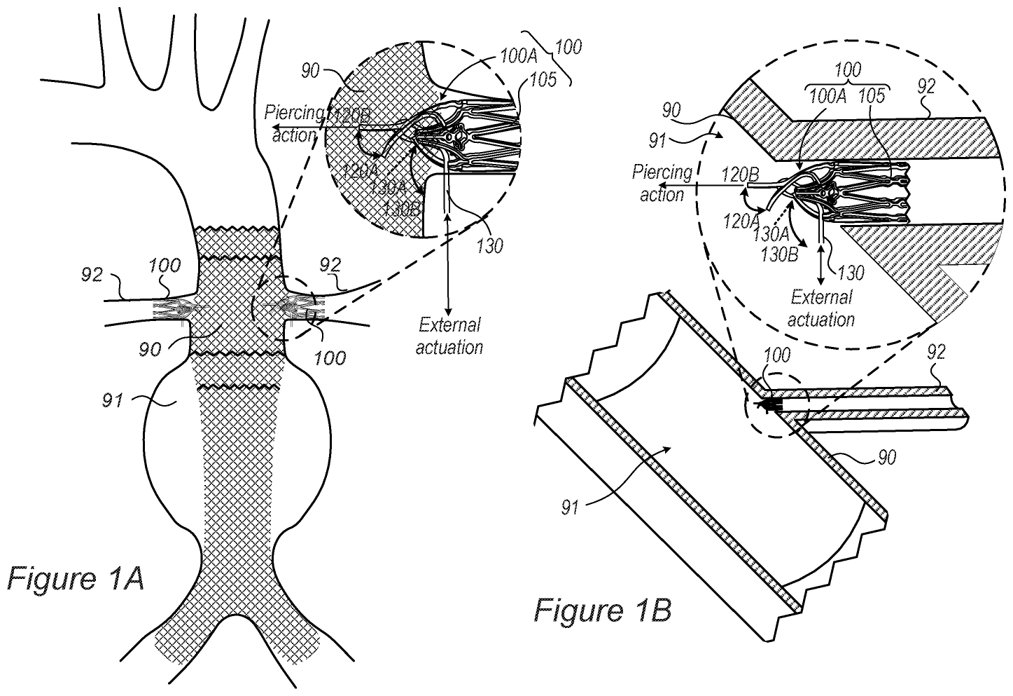

FIGS. 1A and 1B illustrate schematically the attachment of side-branch, piercing stents 100 to elements 90 positioned adjacent to apexes 100A of stents 100, according to some embodiments of the invention. Elements 90 may include an aneurism, a main stent graft introduced into the aneurism and/or blood vessels. Aneurisms comprise localized, blood-filled balloon-like bulges of blood vessels, as illustrated schematically in FIG. 1A with respect to the aorta (aortic aneurysm) and in FIG. 1B with respect to a generic vessel, graft or aneurism. Treatment of aneurism may include supportive elements such as grafts of various kinds that are configured to mechanically support the vessel section and maintain blood flow therethrough. Side-branch stents 100 may be used to support and maintain blood flow through side branches 92 that may be obstructed by the aneurism and/or by grafts inserted to treat them. For example, blocking of side branches 92 with respect to a central volume 91 of the aneurism is a common condition when placing stent grafts to treat aortic aneurism. Side-branch stent(s) 100 may be implanted or inserted into side branches 92 so that their apex(es) are positioned adjacent to respective element(s) 90 such as an aneurism, a main stent graft introduced into the aneurism and/or blood vessels. It is noted that side-branch stent(s) 100 may be perpendicular to element(s) 90 or at an angle thereto (as illustrated schematically in FIGS. 1A and 1B, respectively). Disclosed piercing side-branch stents 100 are configured to be safely and effectively attached to the aneurism, to the main stent graft introduced into the aneurism and/or to the blood vessels, denoted in general as elements 90 (illustrated schematically in FIG. 1A as a stent graft supporting an aneurism, and in FIG. 1B as a blood vessel), and maintain fluid communication of blood between central volume 91 and side branches 92. Further disclosure of the structure and function of piercing side-branch stents 100 is provided below.

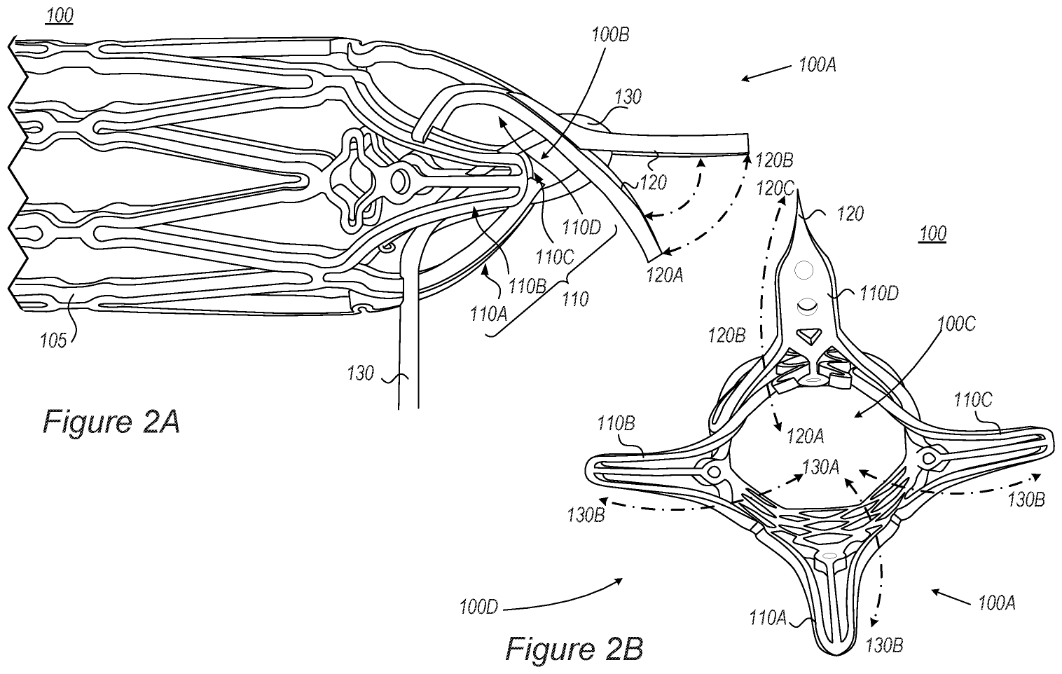

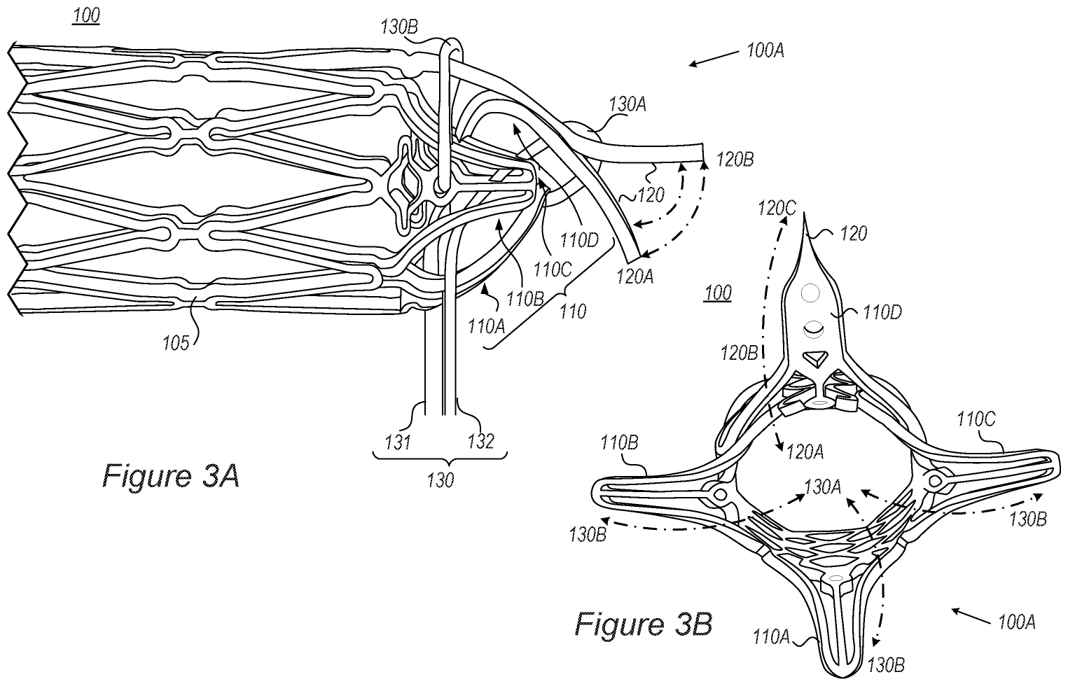

FIGS. 2A and 2B and FIGS. 3A and 3B illustrate schematically side-branch, piercing stents 100, according to some embodiments of the invention. FIGS. 2A and 3A are side views and FIGS. 2B and 3B are perspective front (apical, from the direction of apex 100A) views. Side-branch, piercing stents comprise a stent body 105 (shown partly) having apically at least three movable leaves 110. In the illustrated non-limiting example, there are three non-piercing leaves denoted 110A, 110B, 110C and one piercing leaf 110D, as explained in the following. Leaves 110 are configured to be controllably moveable in a medial direction (denoted schematically by the arrows in FIGS. 2B and 3B) between at least two leaf states comprising (i) a closed state 130A in which an apical stent opening (denoted schematically by the numeral 100B) is smaller than a cross section (denoted schematically by the numeral 100C) of stent 110, and (ii) an open state 130B in which the apical stent opening (denoted schematically by the numeral 100D) is larger than cross section 100C of stent 110. The transition of leaves 110 (e.g., 110A, 110B, 110C) from closed state 130A to open state 130B may be carried out through an opening, or fenestration, created by the piercing leaf(ves) (see explanation below) and may function to anchor the connection of side-branch stent 110 to main element 90 such as main graft stent or blood vessel as shown in FIGS. 1A and 1B.

At least one of leaves (or fingers) 110, in the illustrated, non-limiting case leaf 110D, may be configured to have an apical piercing (penetrating) tip 120 that is controllably moveable between at least two piercing states comprising (a) an extended state 120B (see FIGS. 2A and 3A), in which apical piercing tip 120 is positioned to pierce element 90 positioned adjacent to apex 100A of stent 100, and (b) a contracted state 120A in which apical piercing tip 120 is positioned not to pierce element 90 positioned adjacent to apex 100A of stent 110. It is noted that leaf 110D may be extended further (denoted as state 120C) to anchor side-branch stent 110 to main element 90, as illustrated schematically in FIGS. 2B and 3B and thus have a double function, both as a piercing element and as an anchoring element. FIGS. 1A and 1B also show, in a highly schematic manner, piercing extended state 120B positioned to penetrate element 90 positioned adjacent to apex 100A of stent 100, such as main graft stent 90 in FIG. 1A and blood vessel 90 in FIG. 1B—the piercing action being indicated schematically in both.

Side-branch, piercing stent 100 further comprises at least one controlling member 130 configured to control, mechanically and upon actuation, the movement of at least some of leaves 110 (see schematic “external actuation” in FIG. 1A). For example, controlling member(s) 130 may be further configured to control, mechanically and upon actuation, the movement of apical piercing tip 120 of leaf(ves) 110D, e.g., from contracted state 120A to extended state 120B and possibly further to open state 120C; and may be further configured to control, mechanically and upon actuation, the movement of other leaves 110, e.g., any or all of leaves 110A, 110B, 110C, 110D, e.g., from closed state 130A to open state 130B. Controlling member(s) 130 may be configured to control any of these movements along one or both ways (e.g., from one state to another and possibly back) and may configured to induce abrupt movements between the respective states and/or to control gradual movements between the respective states, controllably enabling intermediate states.

In certain embodiments, controlling member(s) 130 may be configured to coordinate the movement of leaves 110 and the movement of apical piercing (penetrating) tip 120 to enter the tips of leaves 110 (e.g., leaves 110A, 110B, 110C) in closed state 130A into a hole (or fenestration) pierced by apical piercing tip 120 and spread the tips to reach open state 130B of leaves 110 within element 90 (e.g., blood vessel of main graft stent) positioned adjacent to apex 100A of stent 100—to anchor leaves 110 within element 90 and connect side-branch, piercing stent 100 to element 90—providing fluid communication between central volume 91 and side branches 92.

For example, controlling member 130 may comprise comprises one or more strings or wires 130, 131, 132 that are associated with leaves 110 and with apical piercing tip 120 to control their movement. FIG. 2A illustrates schematically single string or wire 130 and FIG. 3A illustrates schematically two strings or wires 131, 132, collectively denoted controlling members 130. For example, controlling member 130 (e.g., wires 130, 132) may be configured to rotate apical piercing tip 120 from contracted state 120A to extended state 120B thereof to pierce element 90 (e.g., upon initial manipulation), and possibly further to extend corresponding leaf 110D into open state 120C (e.g., upon consecutive manipulation of wires 130, 132). Controlling member 130 (e.g., wires 130, 132) may be configured to rotate apical piercing tip 120 in a reversible manner, possibly enabling adjustment or correction of the piercing location if needed. Controlling member 130 (e.g., wires 130, 132) may be configured to move some or all leaves 110 as well from closed state 130A to open state 130B upon the consecutive manipulation thereof, or possibly an additional string or wire 131 (see FIG. 3A) may be configured to control the movement of leaves 110A, 110B, 110C while string or wire 132 may be configured to control the movement of leaf 110D having piercing tip 120. Any of wires 130, 131, 132 may be pulled and released, e.g., for carrying out the initial manipulation of creating the fenestration, and then may be pulled and removed, e.g., for carrying out the consecutive manipulation. In case controlling member 130 comprises two or more strings or wires 131, 132, each of these may be pulled and released to perform its respective operation on leaves 110 of side-branch stent 100. In certain embodiments, first string 132 may be configured to rotate apical piercing tip 120 from contracted state 120A to extended state 120B thereof, and second string 131 may be configured to move leaves 110 from closed state 130A to open state 130B thereof. Strings 131, 132 may be manipulated by pulling out and releasing the respective strings. Controlling member 130 may be configured to provide full control of the movements of tip 120 and leaves 110, to allow an external operator thereof (e.g., of wires 130, 131, 132) to handle the engagement of side branch stent 100 to element 90 in great precision and full control. Controlling member 130 may further comprise restraining elements (not shown, e.g., an additional wire or element connected to one of wires 131, 132) that hold leaves 110 in place until the penetration of tip 120 has been successfully completed, and then release leaves 110 to anchor side branch stent 100 within element 90.

It is noted that the number of leaves or fingers 110 may be one, two, three, four, or more, as required by the design and functionality of stent 100. The movement of apical tip 120 may be rotational, linear or a combination thereof, adapted together with the shape of tip 120 to puncture the side of element 90 upon the respective movement and consecutively anchor leaves 110 and stent 100 within element 90—in a stable manner, at a specified position and maintaining fluid communication blood between central volume 91 and side branches 92. It is noted that apical tip 120 and its movement may be configured to for apex 100A of stent 100 into the opening created by tip 120, so that unfolding leaves 110 occurs within the volume of element 90 to anchor leaves 110 at the inner side of element 90.

Side-branch stents 100 may be made of various material, such as bio-compatible alloys or polymers.

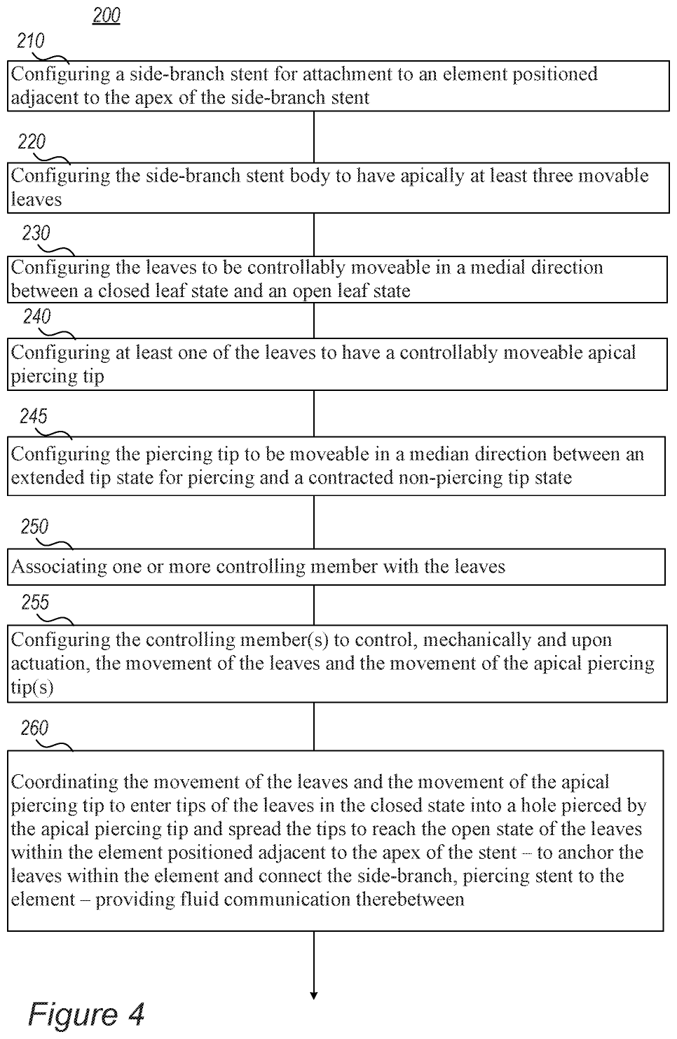

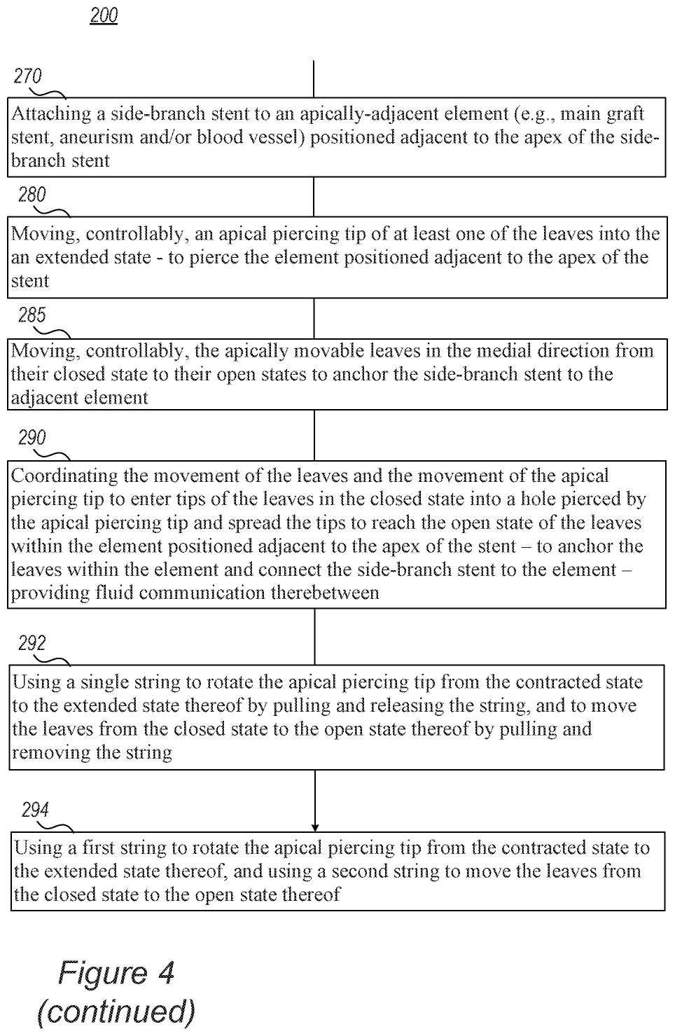

FIG. 4 is a high-level flowchart illustrating methods 200, according to some embodiments of the invention. The method stages may be carried out with respect to side-branch stent 100 described above, which may optionally be configured to implement method 200. Method 200 may comprise the following stages, irrespective of their order.

Methods 200 comprise methods of configuring a side-branch stent for attachment to an element positioned adjacent to the apex of the side-branch stent (stage 210) and methods of attaching the side-branch stent to the element positioned adjacent to the apex of the side-branch stent (stage 270).

Configuration methods 210 comprise configuring the side-branch stent body to have apically at least three movable leaves (stage 220), configuring the leaves to be controllably moveable in a medial direction between at least two leaf states comprising (i) a closed state in which an apical stent opening is smaller than a cross section of the stent, and (ii) an open state in which the apical stent opening is larger than the cross section of the stent (stage 230); configuring at least one of the leaves to have an apical piercing tip that is controllably moveable between at least two piercing states (stage 240) and configuring the tip to be movable between (a) an extended state in which the apical piercing tip is positioned to pierce the element positioned adjacent to the apex of the stent, and (b) a contracted state in which the apical piercing tip is positioned not to pierce the element positioned adjacent to the apex of the stent (stage 245); and associating with the leaves at least one controlling member (stage 250) and configuring controlling member(s) to control, mechanically and upon actuation, the movement of the leaves and the movement of the apical piercing tip of the at least one of the leaves (stage 255).

Method 200 may further comprise coordinating the movement of the leaves and the movement of the apical piercing tip to enter tips of the leaves in the closed state into a hole pierced by the apical piercing tip and spread the tips to reach the open state of the leaves within the element positioned adjacent to the apex of the stent—to anchor the leaves within the element and connect the side-branch, piercing stent to the element—providing fluid communication therebetween (stage 260). For example, the movement of the apical piercing tip may comprise a rotation and/or a reversible rotation thereof to form an opening or fenestration in the adjacent element by the piercing tip—to allow moving the apical tip of the side-branch stent into the element and anchor it thereto by spreading the apical leaves.

Attachment methods 270 comprise moving, controllably, an apical piercing tip of at least one of the leaves into (a) an extended state in which the apical piercing tip is positioned to pierce the element positioned adjacent to the apex of the stent (stage 280), and moving, controllably, at least three apical movable leaves in a medial direction from (i) a closed state in which an apical stent opening is smaller than a cross section of the stent into (ii) an open state in which the apical stent opening is larger than the cross section of the stent (stage 285), and optionally coordinating the movement of the leaves and the movement of the apical piercing tip to enter tips of the leaves in the closed state into a hole pierced by the apical piercing tip and spread the tips to reach the open state of the leaves within the element positioned adjacent to the apex of the stent—to anchor the leaves within the element and connect the side-branch, piercing stent to the element—providing fluid communication therebetween (stage 290).

In various embodiments, the controlling member(s) may comprise a single string and method 200 may further comprise using the single string to rotate the apical piercing tip from the contracted state to the extended state thereof by pulling and releasing the string, and to move the leaves from the closed state to the open state thereof by pulling and removing the string (stage 292); or the controlling member(s) may comprise two strings, and method 200 may further comprise using a first string to rotate the apical piercing tip from the contracted state to the extended state thereof, and using a second string to move the leaves from the closed state to the open state thereof (stage 294). Either or both strings may be manipulated by pulling out and releasing the respective strings, and additional members may be configured to coordinate the movements of the tip(s) and the leaves, e.g., externally at a highly controllable manner and with high precision.

In the above description, an embodiment is an example or implementation of the invention. The various appearances of “one embodiment”, “an embodiment”, “certain embodiments” or “some embodiments” do not necessarily all refer to the same embodiments. Although various features of the invention may be described in the context of a single embodiment, the features may also be provided separately or in any suitable combination. Conversely, although the invention may be described herein in the context of separate embodiments for clarity, the invention may also be implemented in a single embodiment. Certain embodiments of the invention may include features from different embodiments disclosed above, and certain embodiments may incorporate elements from other embodiments disclosed above. The disclosure of elements of the invention in the context of a specific embodiment is not to be taken as limiting their use in the specific embodiment alone. Furthermore, it is to be understood that the invention can be carried out or practiced in various ways and that the invention can be implemented in certain embodiments other than the ones outlined in the description above.

The invention is not limited to those diagrams or to the corresponding descriptions. For example, flow need not move through each illustrated box or state, or in exactly the same order as illustrated and described. Meanings of technical and scientific terms used herein are to be commonly understood as by one of ordinary skill in the art to which the invention belongs, unless otherwise defined. While the invention has been described with respect to a limited number of embodiments, these should not be construed as limitations on the scope of the invention, but rather as exemplifications of some of the preferred embodiments. Other possible variations, modifications, and applications are also within the scope of the invention. Accordingly, the scope of the invention should not be limited by what has thus far been described, but by the appended claims and their legal equivalents.

1. A side-branch, piercing stent comprising:
a stent body having apically at least three movable leaves, the leaves configured to be controllably moveable in a medial direction between at least two leaf states comprising (i) a closed state in which an apical stent opening is smaller than a cross section of the stent, and (ii) an open state in which the apical stent opening is larger than the cross section of the stent, and
at least one controlling member configured to control, mechanically and upon actuation, the movement of the leaves;
wherein at least one of the leaves is configured to have an apical piercing tip that is controllably moveable between at least two piercing states comprising (a) an extended state in which the apical piercing tip is positioned to pierce an element positioned adjacent to the apex of the stent, and (b) a contracted state in which the apical piercing tip is positioned not to pierce an element positioned adjacent to the apex of the stent, and
wherein the at least one controlling member is further configured to control, mechanically and upon actuation, the movement of the apical piercing tip of the at least one of the leaves.

2. The side-branch, piercing stent of claim 1, wherein the at least one controlling member is further configured to coordinate the movement of the leaves and the movement of the apical piercing tip to enter tips of the leaves in the closed state into a hole pierced by the apical piercing tip and spread the tips to reach the open state of the leaves within the element positioned adjacent to the apex of the stent—to anchor the leaves within the element and connect the side-branch, piercing stent to the element—providing fluid communication therebetween.

3. The side-branch, piercing stent of claim 1, wherein the element positioned adjacent to the apex of the stent comprises an aneurism and/or a main stent graft introduced into the aneurism.

4. The side-branch, piercing stent of claim 1, wherein the at least one controlling member comprises at least one string that is associated with the leaves and the apical piercing tip to control their movement.

5. The side-branch, piercing stent of claim 1, wherein the at least one controlling member is configured to rotate the apical piercing tip from the contracted state to the extended state thereof.

6. The side-branch, piercing stent of claims 5, wherein the at least one controlling member is configured to carry out the rotation in a reversible manner.

7. The side-branch, piercing stent of claim 4, wherein the at least one controlling member comprises a single string configured to rotate the apical piercing tip from the contracted state to the extended state thereof upon initial manipulation, and to move the leaves from the closed state to the open state thereof upon consecutive manipulation of the string.

8. The side-branch, piercing stent of claim 7, wherein the initial manipulation comprises pulling and releasing the string and the consecutive manipulation comprises pulling and removing the string.

9. The side-branch, piercing stent of claim 4, wherein the at least one controlling member comprises two strings comprising a first string configured to rotate the apical piercing tip from the contracted state to the extended state thereof, and a second string configured to move the leaves from the closed state to the open state thereof.

10. The side-branch, piercing stent of claim 9, wherein both strings are manipulated by pulling out and releasing the respective strings.

11. A method of configuring a side-branch stent for attachment to an element positioned adjacent to the apex of the side-branch stent, the method comprising:
configuring the side-branch stent body to have apically at least three movable leaves, the leaves configured to be controllably moveable in a medial direction between at least two leaf states comprising (i) a closed state in which an apical stent opening is smaller than a cross section of the stent, and (ii) an open state in which the apical stent opening is larger than the cross section of the stent,
configuring at least one of the leaves to have an apical piercing tip that is controllably moveable between at least two piercing states comprising (a) an extended state in which the apical piercing tip is positioned to pierce the element positioned adjacent to the apex of the stent, and (b) a contracted state in which the apical piercing tip is positioned not to pierce the element positioned adjacent to the apex of the stent, and
associating with the leaves at least one controlling member configured to control, mechanically and upon actuation, the movement of the leaves and the movement of the apical piercing tip of the at least one of the leaves.

12. The method of claim 11, further comprising coordinating the movement of the leaves and the movement of the apical piercing tip to enter tips of the leaves in the closed state into a hole pierced by the apical piercing tip and spread the tips to reach the open state of the leaves within the element positioned adjacent to the apex of the stent—to anchor the leaves within the element and connect the side-branch, piercing stent to the element—providing fluid communication therebetween.

13. The method of claim 11, wherein the movement of the apical piercing tip comprises a rotation and/or a reversible rotation thereof.

14. A method of attaching a side-branch stent to an element positioned adjacent to the apex of the side-branch stent, the method comprising:
moving, controllably, an apical piercing tip of at least one of the leaves into (a) an extended state in which the apical piercing tip is positioned to pierce the element positioned adjacent to the apex of the stent, and
moving, controllably, at least three apical movable leaves in a medial direction from (i) a closed state in which an apical stent opening is smaller than a cross section of the stent into (ii) an open state in which the apical stent opening is larger than the cross section of the stent.

15. The method of claim 14, further comprising using the side-branch stent configured according to the method of claim 11.

16. The method of claim 14, further comprising coordinating the movement of the leaves and the movement of the apical piercing tip to enter tips of the leaves in the closed state into a hole pierced by the apical piercing tip and spread the tips to reach the open state of the leaves within the element positioned adjacent to the apex of the stent—to anchor the leaves within the element and connect the side-branch, piercing stent to the element—providing fluid communication therebetween.

17. The method of claim 14, wherein the element positioned adjacent to the apex of the stent comprises an aneurism and/or a main stent graft introduced into the aneurism.

18. The method of claim 14, wherein the at least one controlling member comprises a single string and wherein the method further comprises using the single string to rotate the apical piercing tip from the contracted state to the extended state thereof by pulling and releasing the string, and to move the leaves from the closed state to the open state thereof by pulling and removing the string.

19. The method of claim 14, wherein the at least one controlling member comprises two strings, and wherein the method further comprises using a first string to rotate the apical piercing tip from the contracted state to the extended state thereof, and using a second string to move the leaves from the closed state to the open state thereof

20. The method of claim 19, wherein both strings are manipulated by pulling out and releasing the respective strings.
